SEQUENCE LISTING (3 sequences)

SEQ ID NO: 1 - protein, 9 aa, Strongylocentrotus purpuratus
CFISNCPKG

SEQ ID NO: 2 - protein, 9 aa, Homo sapiens
CYFQNCPRG

SEQ ID NO: 3 - protein, 9 aa, Homo sapiens
CYIQNCPLG

(19) United States
(12) Patent Application Publication
(10) Pub. No.: US 2025/0074939 A1
(43) Pub. Date: Mar. 6, 2025

(54) SEA URCHIN-DERIVED PEPTIDE AND USE THEREOF

(71) Applicant: NATIONAL MARINE BIODIVERSITY INSTITUTE OF KOREA, Chungcheongnam-Do (KR)

(72) Inventors: Seon Mi Jo, Jeollabuk-Do (KR); Seung Hyun Jung, Chungcheongnam-Do (KR); Hye Suck An, Chungcheongnam-Do (KR); Jei Ha Lee, Daejeon (KR); Kyung Hwa Baek, Chungcheongnam-Do (KR); Hea Bin Kim, Gyeongsangbuk-Do (KR); Ha Jeong Lee, Chungcheongnam-Do (KR)

(21) Appl. No.: 18/720,537

(22) PCT Filed: Dec. 5, 2022
(86) PCT No.: PCT/KR2022/019643

§ 371 Date: Jun. 14, 2024

(30) Foreign Application Priority Data
Dec. 15, 2021 (KR) 10-2021-0179481

(51) Int. Cl. C07K 7/06; A61K 38/00

(52) U.S. Cl. CPC C07K 7/06; A61K 38/00

(57) ABSTRACT

The present invention relates to a sea urchin-derived peptide and a use thereof and more specifically, to a use of the sea urchin-derived peptide for preventing or treating a disease associated with the V1B receptor of Echinotocin, which is a peptide derived from sea urchins. The Echinotocin according to the present invention was found to have an effect of inducing activation of the V1B receptor, which is a type of vasopressin receptors. The V1B receptor, which is a vasopressin receptor, exists in the anterior pituitary gland and the hippocampus CA2 region and is known to increase intracellular calcium concentration and to be involved in a response to stress and sociality. Therefore, the Echinotocin according to the present invention can find various applications in the field of preventing, alleviating, or treating V1B receptor-related diseases.

### TECHNICAL FIELD

The present invention relates to a sea urchin-derived peptide and a use thereof and more specifically, to a use of a sea urchin-derived peptide, Echinotocin for preventing or treating V1B receptor-related diseases.

### BACKGROUND ART

V1B receptors are also called V3 receptors. The V1B receptors were found later than other oxytocin/vasopressin receptors V1AR, V2R, and OXTR. Research on the function of the V1B receptors and receptor activity regulators is still relatively limited compared to other receptors. The V1B receptors are located in the anterior pituitary gland and the hippocampus CA2 region, and are also found in other brain regions and body parts. When the V1B receptor is activated by binding to vasopressin, intracellular signaling mediated by a G protein occurs.

When activated, the V1B receptor exiting in the anterior pituitary gland stimulates the secretion of adrenocorticotropic hormone (ACTH). Mice from which the V1B receptor is removed have lower blood concentrations of adrenocorticotropic hormone (ACTH) and corticosterone than normal mice, and when the mice are stressed, these hormones required to withstand stress do not normally increase.

Single nucleotide polymorphisms (SNPs) in the V1B receptor are known to have resistance to recurrent major depressive disorder (depression), and selective antagonists of the V1B receptor have been studied in relation to the treatment of depression.

Additionally, it has been found that the V1B receptors are also present in other brain regions, including the hippocampus CA2 region. The hippocampus CA2 region is a brain region that plays an important role in recognizing other animals of the same species by animals, distinguishing between familiar and unfamiliar subjects, enabling social memory, and showing social behaviors such as social aggression, social motivation, social preference, and social anxiety.

Male mice from which the V1B receptor is removed had significantly reduced aggression towards other male mice, and the ability to recognize other mice and social motivation were reduced. There were also changes in ultrasonic vocalization, a method of communication with other mice. SSR149415, which is a V1B receptor antagonist, also reduced aggressive responses in hamsters.

When the V1B receptor is re-expressed in mice from which a V1B receptor gene has been removed, sociality-related behaviors are restored. The V1B receptor agonist enhanced excitatory signaling in neurons of the hippocampus CA2 region.

Meanwhile, Strongylocentrotus purpuratus is an animal belonging to the genus Strongylocentrotus, class Echinoidea, and phylum Echinodermata. The Strongylocentrotus purpuratus lives in the eastern Pacific Ocean extending from Mexico to Canada and is deep purple in color and lays orange eggs. The genome of Strongylocentrotus purpuratus was decoded in 2006, and is known to contain approximately 23,300 genes. One type of oxytocin/vasopressin superfamily gene exists in the genome of Strongylocentrotus purpuratus, which was named Echinotocin. The Echinotocin was effective in contracting tube foot and esophageal tissues isolated from Echinus esculentus. However, there has been no previous research on whether the Echinotocin may have physiological activity by acting even on human oxytocin/vasopressin receptors.

### DISCLOSURE

### Technical Problem

Accordingly, the present inventors completed the present invention by confirming a V1B receptor regulatory effect of Echinotocin which was a Strongylocentrotus purpuratus-derived peptide.

Therefore, an object of the present invention is to provide a composition for preventing, alleviating or treating V1B receptor-related diseases, including a peptide represented by an amino acid sequence set forth in SEQ ID NO: 1.

Another object of the present invention is to provide a method for treating V1B receptor-related diseases, including administering a peptide represented by an amino acid sequence set forth in SEQ ID NO: 1 to a subject in need thereof.

### Technical Solution

One aspect of the present invention provides a pharmaceutical composition for preventing or treating V1B receptor-related diseases, including a peptide represented by an amino acid sequence set forth in SEQ ID NO: 1.

Another aspect of the present invention provides a food composition for preventing or alleviating V1B receptor-related diseases, including a peptide represented by an amino acid sequence set forth in SEQ ID NO: 1.

Yet another aspect of the present invention provides a health functional food composition for preventing or alleviating V1B receptor-related diseases, including a peptide represented by an amino acid sequence set forth in SEQ ID NO: 1.

Still another aspect of the present invention provides a method for treating V1B receptor-related diseases, including administering a peptide represented by an amino acid sequence set forth in SEQ ID NO: 1 to a subject in need thereof.

### Advantageous Effects

According to the present invention, Echinotocin was found to have an effect of inducing the activation of the V1B receptor, which is a type of vasopressin receptor. The V1B receptor, which is a vasopressin receptor, exists in the anterior pituitary gland and the hippocampus CA2 region and is known to increase intracellular calcium concentration and to be involved in a response to stress and sociality. Therefore, the Echinotocin according to the present invention can find various applications in the field of preventing, alleviating, or treating V1B receptor-related diseases.

### DESCRIPTION OF DRAWINGS

FIG. 1 is a diagram illustrating results of confirming an effect of activating a human oxytocin/vasopressin receptor by Strongylocentrotus purpuratus-derived Echinotocin according to the present invention.

FIG. 2 is a diagram illustrating results of confirming an effect of inhibiting a human oxytocin/vasopressin receptor by Strongylocentrotus purpuratus-derived Echinotocin according to the present invention.

### BEST MODE OF THE INVENTION

Hereinafter, the present invention will be described in detail.

According to an aspect of the present invention, there is provided a composition for preventing, alleviating or treating V1B receptor-related diseases, including a peptide represented by an amino acid sequence set forth in SEQ ID NO: 1.

In an embodiment of the present invention, the peptide is preferably used for activation of a V1B receptor, but the scope of the present invention is not limited thereto.

In an embodiment of the present invention, the peptide is preferably derived from Strongylocentrotus purpuratus, but the scope of the present invention is not limited thereto.

The peptide represented by the amino acid sequence set forth in SEQ ID NO: 1 is Echinotocin, discovered through transcriptome analysis of Strongylocentrotus purpuratus. The Echinotocin was known to have an effect of contracting the tube foot and esophageal tissues isolated from Echinus esculentus. However, there has been no previous research on whether Echinotocin may have physiological activity by acting even on human oxytocin/vasopressin receptors.

In general, all products produced by evolutionarily conserved homologous genes in different species are called by the same name and considered to have similar functions, but as an exception, since peptides belonging to the oxytocin/vasopressin superfamily may greatly vary in the functions even with one or two differences in the amino acid sequence, if there is even a slight difference in chemical structure, the peptides are given separate names and treated as separate materials.

In an embodiment of the present invention, the Echinotocin represented by SEQ ID NO: 1 may further include an amine group (—NH₂) at the C-terminus, and may have disulfide bonds formed between amino acids at positions 1 and 6.

In the present invention, the peptide refers to a linear molecule formed by binding amino acid residues to each other by peptide bonds. The peptide may be prepared according to a chemical synthesis method known in the art, preferably prepared according to a solid-phase synthesis technique, but is not limited thereto. In addition, the peptide may be extracted from the nature or prepared by genetic recombination and expression systems based on a DNA sequence.

The scope of the present invention includes functional equivalents of the peptide represented by the amino acid sequence set forth in SEQ ID NO: 1.

The functional equivalent refers to a peptide that exhibits substantially homogeneous physiological activity as the peptide represented by the amino acid sequence set forth in SEQ ID NO: 1 as a result of addition, substitution, or deletion of amino acids. Amino acids introduced for addition or substitution may include not only natural amino acids but also unnatural amino acids and optically isomeric amino acids.

The substantially homogeneous physiological activity refers to the activity of the peptide of the present invention, that is, oxytocin/vasopressin receptor regulatory activity, and more preferably, selective promotion of the V1B receptor activity. The range of the functional equivalents of the present invention includes derivatives in which some chemical structures of the peptide are modified while maintaining the backbone of the peptide represented by the amino acid sequence set forth in SEQ ID NO: 1 and the oxytocin/vasopressin receptor regulatory activity. For example, the range of the functional equivalents may include structural changes, cyclization, dimerization, multimerization, and conjugation with other materials such as fluorescent materials to change the stability, storage, volatility, solubility, etc. of the peptide. In addition, the range of the functional equivalents includes some fragments of the peptide that exhibit the homogeneous physiological activity. The range of the functional equivalents is not limited to types of salts to be added during the chemical synthesis of the peptide.

The structural modification of the peptide may be in the form of modifying the N-terminus or C-terminus of the peptide represented by the amino acid sequence set forth in SEQ ID NO: 1 or protecting the peptide with various organic groups in order to protect the peptide from protein-cleaving enzymes in vivo and increase stability. That is, the C-terminus of the peptide represented by the amino acid sequence set forth in SEQ ID NO: 1 is not particularly limited as long as it may be modified to increase the stability, but may be preferably modified with a hydroxyl group (—OH) or an amino group (—NH₂). In addition, the N-terminus of the peptide represented by the amino acid sequence set forth in SEQ ID NO: 1 is not particularly limited as long as it may be modified to increase the stability, but preferably, may be modified or deaminated with a group selected from the group consisting of an acetyl group, a fluorenyl methoxy carbonyl (Fmoc) group, a formyl group, a palmitoyl group, a myristyl group, a stearyl group, and a polyethylene glycol (PEG).

It was confirmed through experiments that the peptide represented by the amino acid sequence set forth in SEQ ID NO: 1 according to the present invention selectively activated only the V1B receptor at a specific concentration, but had little effect on other receptors. Specifically, the concentration at which the peptide represented by the amino acid sequence set forth in SEQ ID NO: 1 selectively activates the V1B receptor is preferably 1 μM or less, and more preferably 10 nM or less. The concentration range that maximizes V1B receptor selectivity may vary depending on an experimental method.

In addition, the peptide represented by the amino acid sequence set forth in SEQ ID NO: 1 according to the present invention may simultaneously inhibit or activate the oxytocin/vasopressin receptors. Specifically, when the concentration of the peptide represented by the amino acid sequence set forth in SEQ ID NO: 1 is 10 to 100 nM, the V1B receptor and a V2 receptor may be activated simultaneously, and when the concentration is more than 100 nM, the effects of activating the V1B receptor and the V2 receptor; and inhibiting an oxytocin receptor and a VIA receptor inhibition are exhibited simultaneously.

According to another aspect of the present invention, there is provided a composition for preventing, alleviating or treating vasopressin 1b receptor (V1B receptor)-related diseases, including a peptide represented by an amino acid sequence set forth in SEQ ID NO: 1.

In an embodiment of the present invention, the peptide represented by the amino acid sequence set forth in SEQ ID NO: 1 preferably includes an amine group (—NH₂) at the C-terminus, but is not limited thereto.

In an embodiment of the present invention, the peptide represented by the amino acid sequence set forth in SEQ ID NO: 1 preferably has disulfide bonds formed between amino acids at positions 1 and 6, but is not limited thereto.

In the present invention, the V1B receptor-related disease refers to a disease that requires the activation of the V1B receptor, which is one of vasopressin receptors. In other words, the V1B receptor-related diseases may be prevented, alleviated, and treated by activating the V1B receptor.

In an embodiment of the present invention, the V1B receptor-related diseases may be one or more mental disorders selected from the group consisting of autism spectrum disorder, schizophrenia spectrum disorder, attention deficit hyperactivity syndrome, bipolar disorder, depression, anthropophobia, avoidant personality disorder, delusional disorder, social anxiety disorder, social phobia, social communication disorder, separation anxiety disorder, reactive attachment disorder, decreased libido, loss of libido, antisocial personality disorder, dissocial personality disorder, separation anxiety disorder, reactive attachment disorder, and panic disorder, but are not limited thereto.

In addition, the V1B receptor-related diseases are preferably one or more diseases selected from the group consisting of pancreatic dysfunction, cerebral edema, and inflammatory bowel disease, but are not limited thereto. It is widely known in the art that the above-mentioned V1B receptor-related diseases are closely associated with the V1B receptor.

The composition for preventing, alleviating or treating the V1B receptor-related diseases according to the present invention may be a pharmaceutical composition, a food composition, or a health functional food composition.

When the composition of the present invention is the pharmaceutical composition for preventing or treating the V1B receptor-related diseases, the pharmaceutical composition of the present invention may further include a pharmaceutically acceptable additive. At this time, the pharmaceutically acceptable additive may be used with starch, gelatinized starch, microcrystalline cellulose, lactose, povidone, colloidal silicon dioxide, calcium hydrogen phosphate, lactose, mannitol, taffy, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar and the like. The pharmaceutically acceptable additive according to the present invention is preferably included in an amount of 0.1 to 90 parts by weight based on the composition, but is not limited thereto.

The pharmaceutical composition of the present invention may be administered in various oral or parenteral formulations during actual clinical administration, but may be prepared using commonly used diluents or excipients, such as fillers, extenders, binders, wetting agents, disintegrants, surfactants, etc., for formulations. Suitable formulations known in the art are preferably used with formulations disclosed in the literature (Remington's Pharmaceutical Science, last, Mack Publishing Company, Easton PA).

Solid formulations for oral administration include tablets, pills, powders, granules, capsules, and the like, and these solid formulations may be prepared by mixing at least one excipient, for example, starch, calcium carbonate, sucrose or lactose, gelatin, and the like. Further, lubricants such as magnesium stearate and talc may be used in addition to simple excipients. In addition, liquid formulations for oral administration may correspond to suspensions, oral liquids, emulsions, syrups, and the like, and may include various excipients, for example, wetting agents, sweeteners, aromatic agents, preservatives, and the like, in addition to water and liquid paraffin which are commonly used as simple diluents.

The formulations for parenteral administration include a sterile aqueous solution, a non-aqueous solution, a suspension, an emulsion, a lyophilizing agent, and a suppository. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

In addition, the dose of the pharmaceutical composition of the present invention may vary depending on a formulation method, an administration method, an administration time, and/or an administration route of the pharmaceutical composition, and vary depending on various factors including the type and degree of a response to be achieved by administration of the pharmaceutical composition, the type, age, body weight, and general health condition, symptoms or severity of a disease, sex, diet, and excretion of a subject to be administered, drugs used simultaneously or separately for the corresponding subject, other composition ingredients, and the like, and similar factors well-known in the field of medicine. In addition, an effective dose for a desired treatment may be easily determined and prescribed by those skilled in the art.

The dose of the pharmaceutical composition of the present invention may be administered, for example, at a concentration of preferably 0.05 to 5 mg/kg, more preferably 0.1 to 0.4 mg/kg, much more preferably 0.2 to 0.35 mg/kg, and even more preferably 0.25 mg/kg, but the dose does not limit the scope of the present invention in any way.

The administration route and the administration method of the pharmaceutical composition of the present invention may be independent of each other, and the method is not particularly limited, and may be any administration route and administration method so long as the pharmaceutical composition may reach a corresponding site.

The pharmaceutical composition may be administered orally or parenterally. The parenteral administration method includes, for example, intravenous administration, intraperitoneal administration, intramuscular administration, transdermal administration, subcutaneous administration, intranasal administration, intrathecal administration, intracerebral administration, or the like.

The pharmaceutical composition of the present invention may be used alone or in combination with surgery, radiation therapy, hormone therapy, chemotherapy, and methods of using biological response modifiers, for prevention or treatment of V1B receptor-related diseases.

The composition of the present invention may be a food composition or health functional food composition for preventing or alleviating V1B receptor-related diseases.

In the present invention, the food refers to food having bioregulatory functions, such as prevention and alleviation of the disease, biodefense, immunity, recovery from illness, aging suppression, etc., and needs to be harmless to the human body when taken for a long period of time.

When the food composition of the present invention is a food additive, the peptide represented by the amino acid sequence set forth in SEQ ID NO: 1 as the active ingredient may be added as it is or used with other foods or food ingredients, and may be appropriately used according to a conventional method. The mixed amount of the active ingredients may be suitably determined according to a purpose of use (prevention, health, or therapeutic treatment). In general, the peptide, which is the active ingredient of the present invention, is added in an amount of 15 wt % or less, preferably 10 wt % or less, based on the raw material when preparing foods or beverages. However, in the case of long-term intake for the purpose of health and hygiene or for the purpose of health control, the amount of the composition may be equal to or lower than the range, and there is no problem in terms of safety, so that the active ingredients may be used even in an amount above the range.

The kind of food is not particularly limited. Examples of the food which may be added with the materials include meat, sausages, bread, chocolate, candies, snacks, confectionery, pizza, ramen, other noodles, gums, dairy products including ice cream, various soups, beverages, tea, drinks, alcohol drinks, vitamin complex, and the like, and include all health functional foods in a general meaning.

The health functional food composition according to the present invention may be in various forms such as health drinks. When the health functional food composition according to the present invention is a health drink form, various flavoring agents, natural carbohydrates, or the like may be added as an additional ingredient like general drinks. The above-mentioned natural carbohydrates may be used with monosaccharides such as glucose and fructose, disaccharides such as maltose and sucrose, natural sweeteners such as dextrin and cyclodextrin, synthetic sweeteners such as saccharin and aspartame, and the like. A ratio of the natural carbohydrates may be generally about 0.01 to 10 g, preferably about 0.01 to 0.1 g per 100 ml of the composition of the present invention.

In addition, the food composition or the health functional food composition of the present invention may include various nutrients, vitamins, electrolytes, flavoring agents, coloring agents, pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, or the like. In addition, the composition of the present invention may include pulps for preparing natural fruit juices, fruit juice beverages or vegetable beverages. These ingredients may be used independently or in combination. Although the ratio of these additives is not greatly important, generally, the ratio thereof is selected in a range of 0.01 to 0.1 parts by weight per 100 parts by weight of the composition of the present invention.

According to yet another aspect of the present invention, there is provided a method for treating V1B receptor-related diseases, including administering a peptide represented by an amino acid sequence set forth in SEQ ID NO: 1 to a subject in need thereof.

In an embodiment of the present invention, the subject may be a subject expected to develop the V1B receptor-related diseases; a subject suffering from the V1B receptor-related diseases; or a subject completely cured of the V1B receptor-related diseases, but is not limited thereto.

Duplicated contents are omitted in consideration of the complexity of the present specification, and terms not defined otherwise in the present specification have the meanings commonly used in the art to which the present invention pertains.

### Modes of the Invention

Hereinafter, the present invention will be described in more detail through Examples. These Examples are just illustrative of the present invention, and it will be apparent to those skilled in the art that it is not interpreted that the scope of the present invention is limited to these Examples.

### Example 1. Discovery of Strongylocentrotus purpuratus-Derived Peptide

Echinotocin, which was a peptide derived from Strongylocentrotus purpuratus, was identified through genome/transcriptome information and genetic information search and prior art search of NCBI.

In the present invention, a peptide was prepared and used based on the genetic information and previously known amino acid sequence of Echinotocin, but the same materials may also be obtained through separation and analysis of extracts of Strongylocentrotus purpuratus. In addition, the same materials may be obtained not only from Strongylocentrotus purpuratus but also from other types of sea urchins.

The sequence of Echinotocin, which was the discovered peptide, was shown in Table 1.

TABLE 1

| | Amino acid sequence of |
|---|---|
| Name | active peptide (N→C) |
| Human vasopressin | CYFQNCPRG |
| (SEQ ID NO: 2) | |
| Human oxytocin | CYIQNCPLG |
| (SEQ ID NO: 3) | |
| Echinotocin | CFISNCPKG |
| (SEQ ID NO: 1) | |

As shown in Table 1, it was confirmed that Echinotocin was different in amino acid sequence from human oxytocin and vasopressin.

### Example 2. Confirmation of Regulatory Effect of Echinotocin on Human Oxytocin/Vasopressin Receptors

Since vasopressin-like materials have different effects depending on which receptor and how the materials act, it is obvious that the effectiveness and effect pattern of Echinotocin in humans can also be seen only through experiments. Therefore, in the present invention, it was tested how various concentrations of Echinotocin act on each of the human vasopressin/oxytocin receptors.

### Experimental Method

In Example, regulatory effects of Echinotocin (SEQ ID NO: 1) on an oxytocin receptor and a vasopressin receptor were confirmed. Echinotocin represented by SEQ ID NO: 1 additionally included an amine group (—NH₂) at the C-terminus, and disulfide bonds were formed between amino acids at positions 1 and 6. Vasopressin receptors were divided into types V1 and V2. The V1a receptor exists in the vascular smooth muscle and increases the intracellular calcium concentration and contracts the blood vessels. The V1b receptor exists in the anterior pituitary gland and the hippocampus CA2 region and increases the intracellular calcium concentration and is involved in a response to stress and sociality. In addition, the V2 receptor exists in the collecting tubule of the kidney and is known to increase the intracellular cAMP concentration and promote water reabsorption in the collecting tubule.

### Experiment on Oxytocin Receptor OXTR Activation/Inhibition Effect by Echinotocin

Human epithelial cells (cell line name: ECV304) overexpressing a recombinant human oxytocin receptor were cultured and treated with Echinotocin for each concentration at room temperature, and then changes in intracellular calcium concentration induced by Echinotocin-oxytocin receptor binding were measured using a calcium dye fluorescence imaging method.

The receptor activation effect was based on a 100% increased response in intracellular calcium concentration that may be caused by 1 μM oxytocin, and the relative size of the response induced when Echinotocin was treated alone without oxytocin was measured.

The receptor inhibition effect was based on a 100% increased response in intracellular calcium concentration that may be caused by 30 nM oxytocin, and the relative size of the response induced when 30 nM oxytocin and Echinotocin were simultaneously treated was measured.

### Experiment on Vasopressin Receptor V1AR Activation/Inhibition Effect by Echinotocin

Chinese hamster ovary cells (cell line name: CHO) overexpressing a recombinant human vasopressin V1A receptor were cultured and treated with Echinotocin for each concentration at room temperature, and then changes in intracellular calcium concentration induced by Echinotocin-vasopressin V1A receptor binding were measured using a calcium dye fluorescence imaging method.

The receptor activation effect was based on a 100% increased response in intracellular calcium concentration that may be caused by 1 μM vasopressin, and the relative size of the response induced when Echinotocin was treated alone without vasopressin was measured.

The receptor inhibition effect was based on a 100% increased response in intracellular calcium concentration that may be caused by 10 nM vasopressin, and the relative size of the response induced when 10 nM vasopressin and Echinotocin were simultaneously treated was measured.

Experiment on vasopressin receptor V1BR activation/inhibition effect by Echinotocin

Rat basophilic leukemia cells (cell line name: RBL) overexpressing a recombinant human vasopressin V1B receptor were cultured and treated with Echinotocin for each concentration at room temperature, and then changes in intracellular calcium concentration induced by Echinotocin-vasopressin V1B receptor binding were measured using a calcium dye fluorescence imaging method.

The receptor activation effect was based on a 100% increased response in intracellular calcium concentration that may be caused by 0.1 μM vasopressin, and the relative size of the response induced when Echinotocin was treated alone without vasopressin was measured.

The receptor inhibition effect was based on a 100% increased response in intracellular calcium concentration that may be caused by 5 nM vasopressin, and the relative size of the response induced when 5 nM vasopressin and Echinotocin were simultaneously treated was measured.

### Experiment on Vasopressin Receptor V2R Activation/Inhibition Effect by Echinotocin

Chinese hamster ovary cells (cell line name: CHO) overexpressing a recombinant human vasopressin V2 receptor were cultured and treated with Echinotocin for each concentration at room temperature for 30 minutes, and then intracellular cAMP concentrations accumulated by Echinotocin-vasopressin V2 receptor binding for 30 minutes were measured using an HTRF fluorescence imaging method. The receptor activation effect was based on a 100% increased response in intracellular cAMP concentration caused by 1 nM vasopressin for 30 minutes, and the relative size of the response induced when Echinotocin was treated alone without vasopressin was measured. The receptor inhibition effect was based on a 100% increased response in intracellular cAMP concentration that may be caused by 0.03 nM vasopressin for 30 minutes, and the relative size of the response induced when 0.03 nM vasopressin and Echinotocin were simultaneously treated was measured.

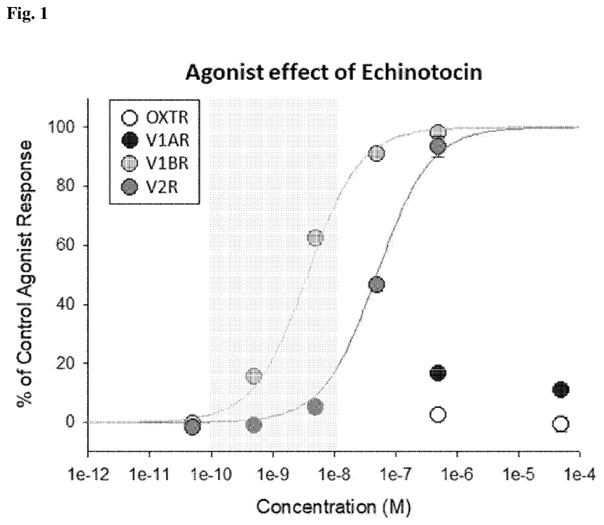

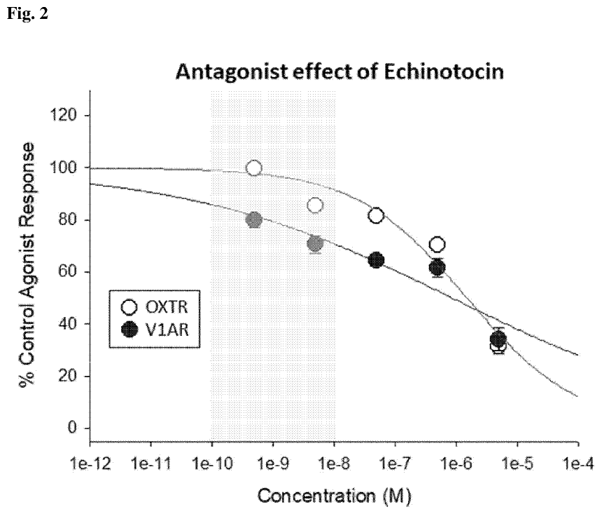

The results of confirming the agonist or antagonist effect of Echinotocin on each receptor were illustrated in FIGS. 1 and 2, respectively.

### EXPERIMENT RESULTS

As illustrated in FIG. 1, it was confirmed that Echinotocin activated the V1B receptor (EC₅₀=49.1 nM) and activated the V2 receptor (EC₅₀=3.68 nM).

As illustrated in FIG. 2, it was confirmed that when the concentration of Echinotocin increased, the V1A receptor activity was able to be inhibited (IC₅₀=1 μM) and the oxytocin receptor activity was also able to be inhibited (IC₅₀=1.7 UM).

Therefore, if Echinotocin was treated at an appropriate concentration (10 nM or less), it can be predicted that Echinotocin selectively activates only the V1B receptor and has little effect on other receptors. Likewise, when changing the concentration of Echinotocin to a high concentration, it is possible to control how much any receptor is activated/inhibited.

The concentration range that maximizes the V1B receptor selectivity may vary depending on an experimental method, but there is a highly industrial availability that only the V1B receptor may be selectively activated at low concentrations of nanomolar or less.

Hereinafter, the present invention will be described in more detail with reference to Preparation Examples. Preparation Examples are only illustrative of the present invention, and the scope of the present invention is not construed to be limited to Preparation Examples.

### Preparation Example 1. Preparation of Pharmaceutical Composition

### 1-1. Preparation of Powders

| | |
|---|---|
| Amino acid represented by SEQ ID NO: 1 | 20 mg |
| Lactose | 100 mg |
| Talc | 10 mg |

The ingredients were mixed and filled in an airtight bag to prepare powders.

### 1-2. Preparation of Tablets

| | |
|---|---|
| Amino acid represented by SEQ ID NO: 1 | 10 mg |
| Corn starch | 100 mg |
| Lactose | 100 mg |
| Magnesium stearate | 2 mg |

The ingredients were mixed and then tableted according to a general tablet preparation method to prepare tablets.

### 1-3. Preparation of Capsules

| | |
|---|---|
| Amino acid represented by SEQ ID NO: 1 | 10 mg |
| Crystalline cellulose | 3 mg |
| Lactose | 14.8 mg |
| Magnesium stearate | 0.2 mg |

The ingredients were mixed and filled into gelatin capsules according to a general capsule preparation method to prepare capsules.

### 1-4. Preparation of Injections

| | |
|---|---|
| Amino acid represented by SEQ ID NO: 1 | 10 mg |
| Mannitol | 180 mg |
| Sterile distilled water for injection | 2974 mg |
| Na₂HPO₄ 2H₂O | 26 mg |

The injections were prepared with the ingredient content per 1 ampoule (2 ml) according to a general method for preparing injections.

### 1-5. Preparation of Liquids

| | |
|---|---|
| Amino acid represented by SEQ ID NO: 1 | 20 mg |
| Isomerized sugar | 10 g |
| Mannitol | 5 g |
| Purified water | Suitable amount |

According to a general method of preparing liquids, each ingredient was added and dissolved in purified water, added with lemon flavor in an appropriate amount, and then the ingredients were mixed, added with purified water to be adjusted to a total of 100 ml, and then filled in a brown bottle and sterilized to prepare liquids.

As described above, specific parts of the present invention have been described in detail, and it will be apparent to those skilled in the art that these specific techniques are merely preferred embodiments, and the scope of the present invention is not limited thereto. Therefore, the substantial scope of the present invention will be defined by the appended claims and their equivalents.

1. A pharmaceutical composition for preventing or treating V1B receptor-related diseases, comprising a peptide represented by an amino acid sequence set forth in SEQ ID NO: 1.

2. The pharmaceutical composition for preventing or treating V1B receptor-related diseases of claim 1, wherein the peptide is used for activating a vasopressin 1b receptor (V1B receptor).

3. The pharmaceutical composition for preventing or treating V1B receptor-related diseases of claim 1, wherein the peptide is derived from Strongylocentrotus purpuratus.

4. The pharmaceutical composition for preventing or treating V1B receptor-related diseases of claim 1, wherein the peptide represented by the amino acid sequence set forth in SEQ ID NO: 1 includes an amine group (—NH₂) at a C-terminus.

5. The pharmaceutical composition for preventing or treating V1B receptor-related diseases of claim 1, wherein the peptide represented by the amino acid sequence set forth in SEQ ID NO: 1 has disulfide bonds formed between amino acids at positions 1 and 6.

6. The pharmaceutical composition for preventing or treating V1B receptor-related diseases of claim 1, wherein the V1B receptor-related diseases are one or more mental disorders selected from the group consisting of autism spectrum disorder, schizophrenia spectrum disorder, attention deficit hyperactivity syndrome, bipolar disorder, depression, anthropophobia, avoidant personality disorder, delusional disorder, social anxiety disorder, social phobia, social communication disorder, separation anxiety disorder, reactive attachment disorder, decreased libido, loss of libido, antisocial personality disorder, dissocial personality disorder, and panic disorder.

7. The pharmaceutical composition for preventing or treating V1B receptor-related diseases of claim 1, wherein the V1B receptor-related diseases are one or more diseases selected from the group consisting of pancreatic dysfunction, cerebral edema, and inflammatory bowel disease.

8. A food composition for preventing or alleviating V1B receptor-related diseases, comprising a peptide represented by an amino acid sequence set forth in SEQ ID NO: 1.

9. The food composition for preventing or alleviating V1B receptor-related diseases of claim 8, wherein the food composition is a dietary supplement composition.

10. A method for treating V1B receptor-related diseases, comprising administering a peptide represented by an amino acid sequence set forth in SEQ ID NO: 1 to a subject in need thereof.
